(19) United States
(12) Patent Application Publication
(10) Pub. No.: US 2007/0167937 A1
(43) Pub. Date: Jul. 19, 2007

(54) Endovascular method and apparatus with electrical feedback

(76) Inventor: Joe Denton Brown, Panama City, FL (US)

Correspondence Address: BACON & THOMAS, PLLC, 625 SLATERS LANE, FOURTH FLOOR, ALEXANDRIA, VA, 22314, US

(21) Appl. No.: 11/714,785

(22) Filed: Mar. 7, 2007

Related U.S. Application Data
(63) Continuation of application No. 11/510,691, filed on Aug. 28, 2006.
(60) Provisional application No. 60/711,273, filed on Aug. 26, 2005.

(51) Int. Cl. A61B 18/18

(52) U.S. Cl. 606/15

(57) ABSTRACT

An apparatus for delivering energy, and in particular laser energy, to a tissue is adapted to minimize or eliminate burn back caused by contact between the energy delivery apparatus and bodily fluids by (i) preventing the energy delivery apparatus from contacting bodily fluids or tissues that might burn or cause the apparatus to burn; and/or (ii) monitoring the apparatus to detect overheating in order to withdraw the apparatus or control the energy supply in case overheating is detected. The apparatus is applicable, by way of example, to treatment of blood vessels using endovascular techniques.

This application is a continuation-in-part of U.S. patent application Ser. No. 11/510,691, filed Aug. 28, 2006, which claimed the benefit of U.S. Provisional Application Ser. Nos. 60/711,273, filed Aug. 26, 2005.

### BACKGROUND OF THE INVENTION

1. Field of the Invention

This invention relates to apparatus for delivering energy to a tissue, and in particular to apparatus for minimizing damage caused by overheating of either the tissue or the energy delivery apparatus by: (i) preventing the energy delivery apparatus from contacting bodily fluids or tissues that might burn or cause the apparatus to burn; and (ii) monitoring the apparatus to detect overheating in order to withdraw the apparatus or control the energy supply in case overheating is detected.

The apparatus of the invention is applicable, by way of example, to treatment of blood vessels using endovascular techniques for delivering laser energy. The apparatus is arranged to prevent an optical fiber from contacting blood in the vessel or the vessel wall, either by completely enclosing the fiber within an introducer sheath, or by passing a liquid through the introducer sheath to flush contaminants away from the end of the fiber.

The overheating detection/feedback apparatus can be used to detect burning tissues or heat given off by the energy delivery apparatus, either through the introducer or through the energy delivery apparatus, with or without an introducer. If an introducer is used, the introducer may act as a waveguide for radiation generated by burning tissues. Alternatively, the clarity of fluid in the introducer may be detected to check for proper flushing or to indirectly detect effects of overheating. In the case of a laser delivery fiber, bends in the fiber can also be detected by monitoring the cladding for light that is captured by the cladding at a bend. Finally, electrical feedback may be included as an alternative to, or in addition, to optical feedback. The electrical feedback may take the form of a thermocouple, thermistor, or other heat-sensitive electrical device, or a photosensor for detecting visible radiation resulting from overheating or burn back.

2. Description of Related Art

U.S. Pat. No. 6,398,777 discloses a method for treating varicose veins, in which a fiber optic line is introduced through an angiocatheter, the vein is emptied of blood using elevation of the limb or other means, and laser energy in wavelengths of 532 to 1064 nanometers is used to damage the entire thickness of the vein wall, causing fibrosis of the blood vessel and thereby causing the blood vessel to decrease in diameter or collapse.

The apparatus used to carry out the method for treating varicose veins is shown in FIG. 1 and includes an optical laser fiber 1, a guide wire (not shown) to place the fiber, ultrasound (not shown) for locating and viewing the fiber as it is situated in a body cavity (vein 2), and an introducer catheter 3 with a hemostasis valve. While this treatment arrangement is effective in treating varicose veins, a problem with the treatment method is that, as the length of the vessel being treated increases, contact between the fiber tip 4 and blood 5 in the vein can cause overheating and burn back of cladding and other buffer materials on the fiber tip, as illustrated in FIGS. 2A and 2B.

In addition to damaging the fiber cladding, burn back can cause continued lasing, charring or carbonization, and weakened fiber integrity. For example, exposing the silica core of a fiber can allow carbonization to the sides of the fiber tip making it weak with the possibility of falling off into the vein. Furthermore, carbonization forming on the distal tip can locally heat the distal fiber tip surface to extreme temperatures sufficient to enough to cause the fiber to start absorbing infrared radiation, thereby causing a thermal runaway that could perforate the vein wall. Still further, charring and/or other effects of overheating or thermal runaway can directly cause negative effects on the patient, such as operative or post-operative pain and/or toxic reactions to compounds resulting from burning or vaporization of materials such as Teflon™. Finally, if the burn back exposes the surfaces on the side of the fiber, then energy is stolen from the core, making the power density lower and effecting the treatment. Despite these problems, however, little has been done to prevent burn back, with the primary focus being to monitor the procedure and replace the fiber or clean the fiber tip before significant burn back occurs.

One solution is disclosed in German Patent Publication DE 31 19372. Since burn back is caused by blood contamination on the distal tip of the fiber, the German publication discloses a protective cap that is placed over the tip of the fiber and that prevents contact with blood. However, fibers with modified tips typically build up char and burn up or have break off failures and therefore this solution is not practical.

Another solution, as noted above, is simply to monitor the procedure. For example, U.S. Pat. No. 5,098,27 discloses a system that monitors pyrolytic glowing of burning tissues at the end of the fiber, while U.S. Pat. No. 6,932,809 monitors radiation emitted by a black body situated adjacent the fiber when the black body is heated. Unfortunately for the patient, by the time that pyrolytic glow or black body radiation is observed, substantial burn back, vein char or perforation may already have occurred. This is especially true of the pyrolytic glow described in U.S. Pat. No. 5,098,427, which can only be transmitted by the laser delivery fiber at wavelengths effectively less than 2 microns. In addition, the inclusion of a separate black body emitter, as disclosed in U.S. Pat. No. 6,932,809, is both inconvenient and expensive.

Finally, U.S. Pat. No. 5,242,438 discloses a laser delivery apparatus that may be suitable for varicose vein treatment, although no such application is disclosed. This patent is of interest for its disclosure of a reflective tip, which may also be used in connection with the present invention. Unlike the tips of the present invention, however, the tip disclosed in U.S. Pat. No. 5,242,438 is specifically intended to be exposed to bodily fluids, and therefore is vulnerable to burn back.

### SUMMARY OF THE INVENTION

It is accordingly a first objective of the invention to provide an apparatus for delivery of energy to a tissue within a patient, in which damage to the energy delivery device is minimized by preventing contamination of the device by bodily fluids that might cause overheating and/or burning of tissues, bodily fluids, or the apparatus itself.

It is a second objective of the invention to provide apparatus for monitoring a surgical procedure involving delivery of energy to tissues in a body cavity, in which overheating can be rapidly and reliably detected with our without the addition of a black body emitter and at any wavelength indicative of such overheating, including visible wavelengths, before or after a pyrolytic glow occurs. It is a third objective of the invention to provide a vascular treatment apparatus and method that prevents blood contamination and burn back, and that can be reliably monitored through an introducer and/or a cladding of a laser delivery fiber.

These objectives are accomplished by modifying the conventional fiber introducer so as to prevent any blood left in the catheter after preparation of the vein for treatment from contacting the fiber tip. The fiber introducer may either be modified to enclose the fiber, in which case laser light is transmitted through the introducer to the treatment area, or the fiber introducer may be arranged such that a liquid in the introducer, for example a saline solution, will flush blood away from the fiber tip. To help prevent contaminants from sticking to the introducer, the introducer is preferably be made of a low friction material such as Teflon, which also has the advantage of permitting smooth drawback of the laser deliver device inside the vein without sticking to tissue or blood.

According to variations of the invention, a fiber radial diffusing tip, which redirects and lowers power density, may be used to fire directly through the laser introducer sheath. Alternatively, ball tips, cones, metal reflectors, mirrors, diffusers, fiber modulation, etc. may be used.

If the introducer is completely closed off at the distal end, the introducer may advantageously include a highly flexible or floppy tip and/or may be rounded off to eliminate the need for a guide wire, saline flush, and hemostasis valve. Alternatively, the sheath may be provided at the distal end with a reduction means or septum to prevent or minimize blood from entering the catheter, but still allow for a guide wire to pass through the introducer sheath and the reduced end or septum. In that case, means for introducing saline may also be allowed to clean the catheter internal diameter from blood residue.

In an especially advantageous embodiment of the invention, burn back is monitored by detecting light exiting the introducer or fiber cladding. The introducer may, for example, act as a waveguide for visible or near visible light emissions resulting from the burn back. If a fluid (i.e. saline) flush is used, an aiming beam may be directed through the fluid in order to measure the clarity of the fluid and, indirectly, by-products of burn back. Moreover, if the cladding is monitored, then bends in the fiber can also be detected based on light that leaks to and is captured by the cladding at the bend.

Alternatively, overheating or burn back may be monitored by an electrical sensor such as a thermocouple, thermistor, or other heat sensitive electrical device, or a photodetector, positioned in the introducer and electrically rather than optically connected to the feedback circuitry.

The detector of the preferred embodiments may of course be used with apparatus that include an introducer of the type described above, as well as with conventional apparatus in which the fiber tip is exposed. In case the fiber tip is exposed, a heat sink or shield may still be added to help prevent burn back.

### BRIEF DESCRIPTION OF THE DRAWINGS

FIG. 1 is a schematic drawing of a conventional varicose vein treatment apparatus.

FIGS. 2A and 2B are side view of an optical fiber, showing the effects of burn back.

FIG. 3 is a schematic drawing of a treatment apparatus constructed in accordance with the principles of a preferred embodiment of the invention.

FIGS. 4A-4F show various fiber tips for use in connection with the preferred treatment apparatus.

FIG. 5 shows a detector that may be used in connection with a treatment apparatus, according to another preferred embodiment of the invention.

FIG. 6 is a schematic drawing of an optical fiber with a heat sink according to another preferred embodiment of the invention.

### DETAILED DESCRIPTION OF THE PREFERRED EMBODIMENTS

As illustrated in FIG. 3, the apparatus of a preferred embodiment of the invention includes an energy delivery device in the form of an optical fiber 10 that is introduced into the vessel 11 by means of a Teflon_ introducer 12. The Teflon introducer lacks a homeostasis valve and has a closed end 13 that extends beyond the tip 14 of the fiber 10 and that has an end 15 arranged to prevent contamination of the fiber tip 14 by blood 11 in the vessel. The Teflon introducer may optionally include a motorized fiber pull back 16. In addition, a detector 17 may be included, as described in more detail below.

End 15 of the introducer may be either open or closed. If it is completely closed, then the end may including cooling means within the introducer for cooling the fiber. Cooling means for use in a catheter are well known and the details of the cooling means do not form a part of the present invention. Alternatively, the end 15 of the introducer may be open, in which case saline or other irrigation fluid may be introduced at the opposite end to flush the end of the introducer and prevent blood or tissue from accumulating there. In either case, blood is prevented from contacting the tip 14 of the fiber, thereby reducing burn back.

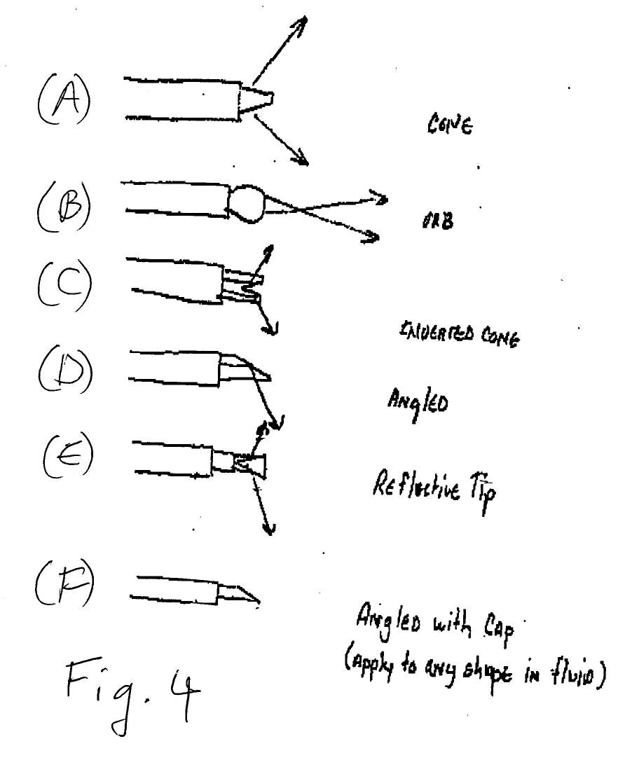

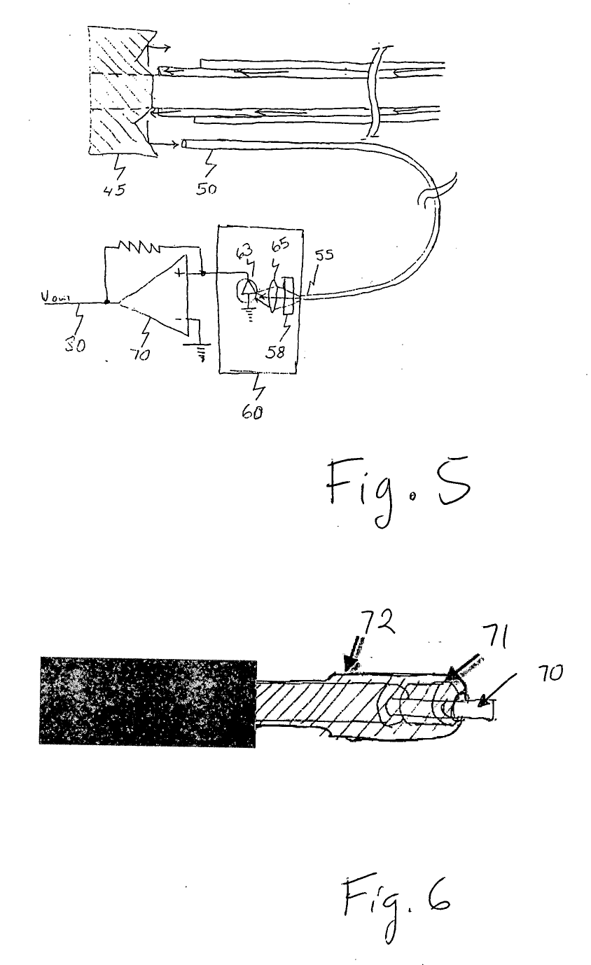

FIGS. 4A to 4F show various tip configurations. The tip can be arranged to direct laser light in a radial direction so as to impinge on walls of the vessel, either in a single direction or along an arc extending up to 360°around the fiber. Such side firing fiber optic tips are known, and the invention is not limited to any particular tip. FIG. 4A shows a conical tip; FIG. 4B shows an orb-shaped tip; FIG. 4C shows an inverted cone-shaped tip; FIG. 4D shows an angled tip; FIG. 4E shows a reflective tip; and FIG. 4F shows an angle tip. The cap of FIG. 4F may be similar to the one shown in U.S. Pat. No. 5,242,438, except that the fiber tip is not exposed. Those skilled in the art will appreciate that a 360° effect may be achieved with a simple side firing laser by rotating the fiber.

FIG. 6 shows a further variation of the invention, used in cases where the bare fiber is exposed, or to further protect the fiber within the introducer. In the variation illustrated in FIG. 6, the bare fiber 70 is surrounded by an optional insulator 71 and heat sink 72, which helps prevent burn back by removing or directing heat away from the fiber tip. Those skilled in the art will appreciate that the insulator and heat sink may be added to a fiber to help prevent burn back even when the fiber is used without an introducer.

In addition to protecting the fiber from contamination and resulting damage, the invention provides for monitoring and detection of conditions that might affect operation of the apparatus, such as burn back or bends in the fiber or energy delivery device. The monitoring device may be a conventional detector 17, but instead of configuring the detector to monitor light exiting the fiber, the detector 17 is configured to detect light exiting the introducer, as indicated by dashed line 18 in FIG. 3. A mirror 19 or fiber may be positioned to direct light exiting the introducer to the detector 17.

Alternatively, detector 17 may be electrically connected to one or more electrical sensors 20 positioned in the introducer to detect heat or light resulting from burn back, and connected to the monitor 17 by a wire, indicated in FIG. 3 by dashed line 21. The electrical sensor may be a thermocouple, thermistor, or any other electrical detector capable of being positioned in the introducer and of generating an electrical signal in response to heat, or a photodetector or similar device sensitive to light generated during overheating or burn back. Although a wire is illustrated, the sensor(s) 21 in the introducer may also include a wireless transmitter or electrical to optical converter for wireless or optical communications.

In embodiments where light exiting the introducer is to be detected, the introducer must act as a waveguide. The inner diameter and material of the introducer may be selected accordingly, depending on the wavelengths of light to be detected. For example, burn back can be directly detected based on light emitted by the burning body fluid, or by reflecting an aiming beam of the laser back to through the introducer to measure the clarity of fluid used to flush the end of the introducer in case of an open-ended sheath.

Alternatively, instead of monitoring light propagating through the introducer, the detector may be arranged to monitor the fiber cladding. An example of a detector capable of monitoring the fiber cladding is illustrated in FIG. 5. In this embodiment, a reflector 45 reflects a portion of the secondary source of radiation 40 into the proximal end of sense fiber 50. The secondary radiation is further transmitted toward the distal end of the sense fiber 55, which directs the radiation to an optical filter 58, after which the light is focused onto a photodetector 63 with a condensing lens 65. The photodetector converts optical radiation to an electrical signal that is further amplified by an op-amp 70. The amplified electrical signal 80 Vout can now be used to control the laser and/or produce a signal to alert the operator of the potential fiber fault.

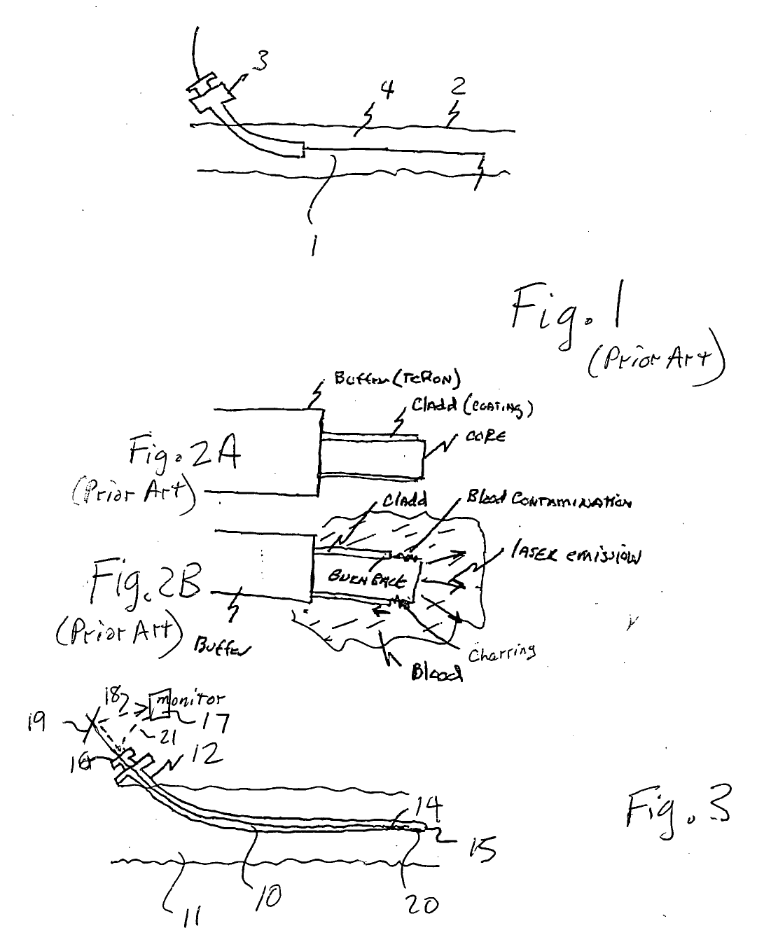

A method of treating varicose veins using the apparatus illustrated in FIGS. 1-6 will now be described. According to the method of the invention, the right/left lower extremity to be treated is prepped and draped in the customary sterile fashion. A layer of ultrasound transmission gel is applied to an ultrasound probe, which is then draped in a sterile sleeve and placed onto the sterile field. Using the ultrasound, the entire greater saphenous vein is mapped with a sterile surgical marker and measurements of its length, maximal and minimal diameters, and tortuosities are recorded.

Under local anesthesia and using ultrasound guidance, percutaneous entry is made with a 19-gauge needle into the Greater or Lesser Saphenous vein. A 0.035″ J tip guide wire is inserted through the needle and is advanced up the Greater Saphenous Vein (GSV) to the Sapheno Femoral Junction (SFJ). The needle is removed and discarded, the skin at the entry site is nicked with a scalpel, and a 45 cm 4 or 5 French introducer sheath is inserted through this opening over the guide wire. With this new procedure the fiber is not required to pass beyond the introducer, the distal introducer tip can be reduced to the same size as the dilator and thus eliminate the need for the dilator. The tip of the introducer should be marked (radiopague and color markings) to indicate the distal end. Also the fiber should have markings (i.e. cm, mm inches . . . ) To indicate its position relative to the introducer, since, the fiber and introducer does not require a dilator. The fibers markings could also be encoded to allow for electrical (i.e. magnetic bar code) or optical means to enable remote control or other feedback. Remote control could include laser enable/disable, automatic pull back, presently a foot switch is used to enable the laser, where a hand switch coupled to the fiber would be with the feedback determining the rate of pullback, laser enable, cumulative joules, etc. . .

The sheath is advanced to the SFJ. The dilator, if used, and the guidewire are then removed, and the laser fiber is introduced to the distal end of the introducer. The distal end of the introducer I.D. should be large enough to pass a guidewire but small enough to prevent the fiber tip from passing thru. Also the fiber should have a mechanical stop that controls how far the fiber can be introduced into the introducer.

As noted above, the fiber tip may be shaped (cone, angled, etc. see attachment A) or capped with a reflective tip to provide more lateral energy perpendicular to the axis of the fiber. The more perpendicular the energy is toward the laser catheter wall the more that is transmitted to the tissue. If power density is an issue, then a diffusing tip or automated fiber movement could also be added. Coaxial water flow may be added where deeper tissue penetration and/or reduced surface reflections from the laser sheath are required.

The position of the fiber and introducer sheath within the GSV is confirmed with ultrasound. The distal end of introducer sheath, is then positioned one or two centimeters below the SFJ. While preventing the introducer sheath from moving, the fiber is withdrawn.

The location of the introducer is confirmed using ultrasound anesthesia is administered along the greater saphenous vein. A final check of the introducer position, about 1 to 2 cm below the SFJ is made using ultrasound and by direct visualization of the red aiming beam through the skin. Anesthesia, is administered along the greater saphenous vein. A final check of the fiber position, about 1 to 2 cm below the SFJ is made using ultrasound and protective eyewear is worn by all persons in the operating room. The laser source, such as but not limited to 810,940 or 980 nm, is turned on by means of a foot pedal or hand piece activation switch. Using continuous energy, e.g., 14 watts, the fiber is withdrawn at a rate of 1 to 2 mm/second. Because the laser sheath is not removed while treating, vein areas needing further treatment can be retreated by repositioning the fiber within the introducer to the desired treatment area. Afterwards, both the laser fiber and sheath are removed.

Repeat ultrasound imaging is performed to confirm absence of flow through the entire length of treated vein, and absence of deep venous thrombosis immediately and 5 minutes following the procedure. After assuring hemostasis, the skin incision over the saphenous vein is closed with a bandage. A class 2-compression stocking is placed on the leg of the treated vein.

Having thus described a preferred embodiment of the invention in sufficient detail to enable those skilled in the art to make and use the invention, it will nevertheless be appreciated that numerous variations and modifications of the illustrated embodiment may be made without departing from the spirit of the invention, and it is intended that the invention not be limited by the above description or accompanying drawings, but that it be defined solely in accordance with the appended claims.

What is claimed is:

1. Apparatus for therapeutic in vivo application of energy to a tissue, comprising:
an optical fiber;
a catheter arranged to be inserted into a body cavity and to receive said optical fiber, said optical fiber being moved through said catheter to a position at which energy may be delivered by said optical fiber to the tissue; and
a feedback circuit including a detector positioned in said catheter for detecting overheating or burn back of at least one of said optical fiber, said tissue, and said catheter.

2. Apparatus as claimed in claim 1, wherein said detector is a thermal sensor.

3. Apparatus as claimed in claim 2, wherein said thermal sensor is a thermocouple.

4. Apparatus as claimed in claim 2, wherein said theremal sensor is a thermistor.

5. Apparatus as claimed in claim 1, wherein said detector is an optical detector.

6. Apparatus for therapeutic in vivo application of energy to a tissue, comprising:
an energy delivery device;
an introducer arranged to be inserted into a body cavity and to receive said energy delivery device, said energy deliver device being moved through said introducer to a position at which energy may be delivered to the tissue; and
a detector for detecting radiation transmitted back through said introducer, said introducer acting as a waveguide for transmitting said radiation.

7. Apparatus as claimed in claim 5, wherein said radiation is visible light.

8. Apparatus for therapeutic in vivo application of energy to a tissue, comprising:
an energy delivery device;
an introducer arranged to be inserted into a body cavity and to receive said energy delivery device, said energy deliver device being moved through said introducer to a position at which energy may be delivered to the tissue; and
a detector for detecting radiation transmitted back through a fluid in said introducer in order to detect a clarity of said fluid.

9. Apparatus as claimed in claim 8, wherein said radiation is an aiming beam.
